(19) United States
(12) Patent Application Publication
(10) Pub. No.: US 2018/0169071 A1
(43) Pub. Date: Jun. 21, 2018

(54) NUTRITIONAL SUPPLEMENT COMPOSITION

(71) Applicant: Medea Juhasz, New York, NY (US)

(72) Inventor: Medea Juhasz, New York, NY (US)

(21) Appl. No.: 15/472,269

(22) Filed: Mar. 29, 2017

Related U.S. Application Data
(60) Provisional application No. 62/436,670, filed on Dec. 20, 2016.

(51) Int. Cl. A61K 31/4188; A61K 36/258; A61K 36/288; A61K 36/481; A61K 36/38; A61K 36/87; A61K 36/55; A61K 36/05; A61K 36/9066; A61K 9/48; A61K 33/10; A61K 31/095

(52) U.S. Cl. CPC A61K 31/4188; A61K 36/258; A61K 36/288; A61K 36/481; A61K 36/38; A61K 31/095; A61K 36/55; A61K 36/05; A61K 36/9066; A61K 9/48; A61K 33/10; A61K 36/87

(57) ABSTRACT

The present invention is a combination formulation of nutrient ingredients that have been selected and combined in quantities and concentrations that offer human nutritional support as shown in various literature. The present invention, also known as CATALYST GOLD, combines herbal, mineral, vitamin, and superfood supplements to help create a catalyst effect within the body to assist lasting permanent lifestyle and bodily changes. The selected nutrient ingredients provide nutritional support for a broad variety of conditions for example, individuals who are depleted due to chemotherapy, drug addiction, and general poor diet. The present invention also provides an easily orally dosed combination of nutrient ingredients in a small dosage form. The present invention may also be conveniently taken throughout the day to maximize support and ingredient quantities through repeat dosing.

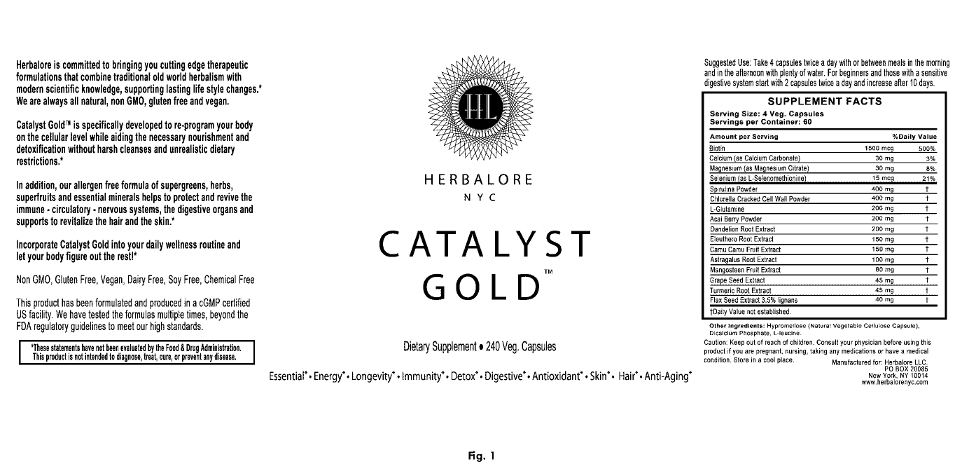

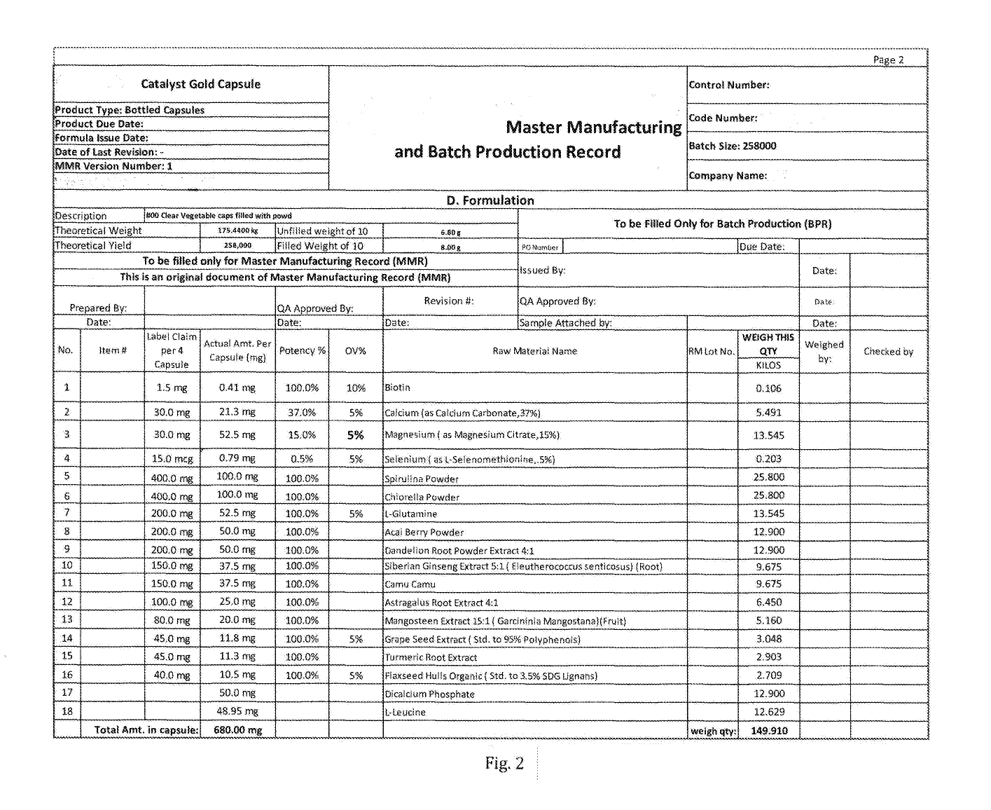

### CROSS-REFERENCE TO RELATED APPLICATIONS

This application claims the benefit of priority to Provisional Application Ser. No. 62/436,670 filed Dec. 20, 2016.

### STATEMENT REGARDING FEDERALLY FUNDED RESEARCH

Not Applicable

### FIELD OF THE INVENTION

The present invention disclosed herein relates to oral compositions of combinations of ingredients comprising dried whole or extracted herbs and fruits, amino acids, essential minerals and vitamins, that offer protection, support, and revitalization of health.

### BACKGROUND

Single herbs, fruits, amino acids, essential minerals, vitamins, and other dietary supplement ingredients have been used individually or in various combinations to provide nutritional support for individuals. Formulas that have been designed in the past are tailored to provide nutrients for a narrow target of nutritional concerns, such as healthy blood sugar, metabolism support, maintenance of bone and connective tissue. However, a nutrient supplement formulation that is a combination of the ingredients in the preset invention and which is intended to support a variety of healthy body functions and which is dosed easily is lacking in the prior art.

The present invention provides an easy to consume oral dose form that provides a wide range of nutrients for the promotion of healthy body systems.

### SUMMARY OF THE INVENTION

The present invention is a combination formulation of nutrient ingredients which have been selected and combined in quantities and concentrations that offer human nutritional support as shown in various literature. The present invention, also known as CATALYST GOLD, is specifically developed to re-program body systems on a cellular level while providing necessary nourishment and aiding detoxification of a subject without harsh digestive system cleanses and difficult dietary restrictions. The present invention, CATALYST GOLD, provides support in boosting the body's natural ability to minimize unhealthy cravings and increase energy levels. The present invention combines mineral, vitamin, herbal, and natural superfood supplements to help create a catalyst effect within the body to assist in achieving lasting permanent lifestyle and bodily changes. The present invention contains an allergen-free formulation of supergreens, herbs, superfruits, vitamins, and essential minerals to help protect and revive the immune system, circulatory system, the nervous system, the digestive organs, and supports revitalization of the hair and the skin. The nutritional support benefits associated with the individual ingredients of the present invention are included in the Detailed Description. The selected nutrient ingredients provide nutritional support for a broad variety of conditions for example, individuals who are depleted due to chemotherapy, drug addiction, and general poor diet. The present invention also provides an easily orally dosed combination of nutrient ingredients in a small dosage form. The present invention may also be conveniently taken throughout the day to maximize support and ingredient quantities through repeat dosing.

### DETAILED DESCRIPTION OF THE INVENTION

The following descriptions are considered to be illustrative of the principles of the present invention and are not intended to be limiting. One of skill in the art will recognize and understand that there are suitable modifications and equivalents that may be used which fall within the scope of the invention described herein. The use of singular forms “a,” “an,” and “the” include plural references unless the context clearly requires otherwise. The embodiments are not limited to those illustrated in the drawings. It should also be understood that the drawings are not necessarily to scale. In certain instances, details may have been omitted that are not necessary for an understanding of the embodiments disclosed herein, for example, conventional fabrication and assembly.

The nutrient supplement ingredients of the present invention individually contain naturally occurring components that are beneficial to human and animal health described and supported herein. The present invention is a convenient combination of amino acids, essential minerals, vitamins, and allergen-free dried ground or extracted herbs and fruits, supergreens, and superfood supplements that help the body to catalyze metabolic reactions, enzymes, and other beneficial effects and act as antioxidants to reactive oxygen species within the body to assist in achieving improved health and permanent lifestyle changes. The ingredients have been shown to support the health of the immune system, circulatory system, the nervous system, the digestive organs, and bones, muscles, hair, and skin.

The nutrient supplement ingredients of the present invention include the following amino acids, essential minerals, vitamins, and allergen-free dried ground or extracted herbs and fruits, supergreens, and superfood supplements.

Biotin, also known as vitamin B7 and vitamin H, is a vital part of a healthy metabolism, creating enzymes, supporting and strengthening the skin, hair, nails, nerves, and the digestive tract. The body utilizes biotin to metabolize carbohydrates, fats, and amino acids according to researchers at the University of Maryland Medical Center. Trueb, R. M. (2016). Serum Biotin Levels in Women Complaining of Hair Loss. Int J Trichology. April-June; 8(2): 73-77. doi: 10.4103/0974-7753.188040

Calcium occurs in nature as limestone, chalk, marble, dolomite, aragonite, or calcite. It is a nutrient mineral that is required and used by the body to maintain healthy bones, muscles, nervous system, and heart. In addition, a salt of calcium called calcium carbonate can provide an added feature of help in relieving intermittent, infrequent heartburn and indigestion according to the American Pharmacists Association. Tytgat, G. N., et al. (2003). Contemporary understanding and management of reflux and constipation in the general population and pregnancy: a consensus meeting. Alimentary Pharmacology & Therapeutics. 18: 291-301. doi:10.1046/j.1365-2036.2003.01679.x

Magnesium is an important mineral that supports bone health, the body's energy production, overall glucose balance, relaxation, and muscle function. Magnesium citrate is a bioavailable form of magnesium that can promote cardiovascular health and calming effects. The citrate form of magnesium is easily digested and better absorbed than other forms of magnesium. Magnesium salts also support regular bowel movement and has been recommended to be taken in doses of from about 30 mg to about 60 mg/per serving for a 70 kg. adult and may be increased or decreased relative to one's weight and may be decreased for infants and the elderly. Volpe, S. L. Magnesium in Disease Prevention and Overall Health (2013). Adv Nutr May 2013, vol. 4:378S-383S. doi: 10.3945/an.112.003483

Selenium plays a key role in the body's detoxification system. It works together with iodine to keep thyroid function strong and consistent according to the US National Library of Medicine. Selenium's re-dox reaction states and antioxidant properties regenerate vitamins E and C, thereby potentially decreasing aging of the skin. Selenium also has positive antiviral effects, and it is essential for successful male and female fertility and reproduction. Baffin, E. E. et al. (2009). Antioxidant Activity of Sulfur and Selenium: A Review of Reactive Oxygen Species Scavenging, Glutathione Peroxidase, and Metal-Binding Antioxidant Mechanism. Cell Biochem Biophys Acta, September 2009, Volume 55, Issue 1, pp 1-23. doi:10.1007/s12013-009-9054-7

Spirulina is a natural algae, contains vitamins B1, B2, B3, B6, B9, vitamin C, vitamin D, vitamin A and vitamin E. It is an excellent source of potassium, calcium, chromium, copper, iron, magnesium, manganese, phosphorus, selenium, sodium, and zinc. Spirulina is also high in omega 3 fatty acids, iron and calcium with over 26 times the calcium in milk. It has a net protein utilization rate of between 55-65% aiding digestion, weight loss, and may help to ease anxiety or depression. In 1974, the World Health Organization described spirulina as “an interesting food for multiple reasons, rich in iron and protein, and is able to be administered to children without any risk,” considering it “a very suitable food.” The United Nations established the Intergovernmental Institution for the use of Micro-algae Spirulina against Malnutrition in 2003. In the late 1980s and early 1990s, both the National Aeronautics and Space Administration (NASA) and the European Space Agency proposed spirulina as one of the primary foods to be cultivated during long-term space missions. Marzieh, S. et al. (2011). The Potential Health Benefits of Algae and Micro Algae in Medicine: A Review on Spirulina platensis. Current Nutrition & Food Science, Volume 7, Number 4, November 2011, pp. 279-285(7). doi: 10.2174/157340111804586457

Chlorella is a single-celled alga that contains the highest amount of chlorophyll of any known plant. It is thought to boost the immune system and help fight infection. It may be useful to treat constipation, fibromyalgia, high blood pressure and high cholesterol. In Japan, it is traditionally used as a treatment for duodenal ulcers, gastritis, hypertension, diabetes, hypoglycemia, asthma, and constipation. More than 20 vitamins and minerals are found in Chlorella, including iron, calcium, potassium, magnesium, phosphorous, pro-vitamin A, vitamins B1, B2, B2, B5, B6, B12, C, E, and K, biotin, inositol, and folic acid. In addition, it is comprised of 60% protein and 18 different amino acids. Research conducted in Japan suggests that Chlorella may have antitumor activity against breast cancer. However, its main use in cancer therapy is to help remove radioactive particles from the body after radiation treatment. Studies in mice have shown that Chlorella vulgaris can protect against the development and spread of cancer. Other rodent studies have shown that it lowers cholesterol and helps organisms eliminate toxic chemicals, such as dioxins. Clinical trials in humans have produced data that indicates that daily dietary supplementation with Chlorella may reduce high blood pressure, lower serum cholesterol levels, accelerate wound healing, and enhance immune functions. Lubitz, J. A. (1963). The Protein Quality, Digestibility, and Composition of Algae, Chlorella 71105. Journal of Food Science, 28: 229-232. doi:10.1111/j.1365-2621.1963.tb00189.x

According to the University of Maryland Medical Center, l-glutamine supports detoxification, wound healing, boosts strength and has an important role in repairing the skeletal muscles. It also supports the improvement of fat reduction and the building of lean muscle. L-glutamine helps to correct acid-alkaline tissue imbalances and diminishes lactic acid. This amino acid may reduce sugar and alcohol cravings by helping the body to suppress insulin and thereby stabilizing blood sugar levels. Bollhalder, L. (2012). A systematic literature review and meta-analysis of randomized clinical trials of parenteral glutamine supplementation. Clinical Nutrition, Volume 32, Issue 2, 213-223. doi:10.1016/j.clnu.2012.11.003

Acai (Euterpe oleracea) berries are high in omega 9 fatty acids as well as in anthocyanins, a form of antioxidants that may lower cholesterol levels in the bloodstream. According to a study published in the June 2008 issue of the Journal of Agricultural and Food Chemistry, acai is a source of antioxidants, rich in Vitamin C, Vitamin E, fiber, protein, calcium, magnesium, potassium, and zinc. Acai may aid to combat fatigue and exhaustion and suppresses inflammation. It may benefit the aging process and support weight loss by boosting both metabolism and energy levels. Mertens-Talcott, S. U., et al. (2008). Pharmacokinetics of Anthocyanins and Antioxidant Effects after the Consumption of Anthocyanin-Rich Acai Juice and Pulp (Euterpe oleracea Mart.) in Human Healthy Volunteers. Journal of Agricultural and Food Chemistry 2008: 56 (17), 7796-7802. doi: 10.1021/jf8007037

Dandelion (Taraxacum officinale) may help detoxify the liver and improve skin and eye conditions. It may also benefit immune responses and is considered to be a detoxifying agent. According to the University of Maryland Medical Center, the leaf contains potassium and can alleviate bloating by removing excess fluid from the body, it acts as a diuretic and increases urine output. Sweeney, B., et al. (2005). Evidence-Based Systematic Review of Dandelion (Taraxacum officinale). Natural Standard Research Collaboration Journal of Herbal Pharmacotherapy Vol. 5, Iss. 1, 2005. doi: 10.1080/J157v05n01_09

Ginseng (Panax ginseng or Panax quinquefolius), and Siberian ginseng (Eleutherococcus senticosus), are known as adaptogens that improve physical performance. Ginseng is traditionally an ethno-botanical that is still used to prevent colds and flu and increase energy, longevity, and vitality. Siberian ginseng, may provide sustained energy, strengthen the memory, help the body adapt to stress and make stress responses more efficient. Unlike many other invigorating plants, Eleutherococcus also shows value as a sleep aid. Siberian Ginseng may be useful as a preventive measure during cold and flu season. Eleutherococcus was discovered in China over 200 years ago. Russian scientist studied the herb after it was brought into space by Soviet astronauts. Dr. B. Tabachnik, a former Soviet sports nutrition researcher and coach, studied Eleutherococcus and administered the herb to athletes. Dr. Tabachnik attributes Eleutherococcus in helping the Soviet Olympic team dominate in the 1988 Olympics. Baranov, A. I. (1982). Medicinal uses of ginseng and related plants in the Soviet Union: Recent trends in the Soviet literature. Journal of Ethnopharmacology, Volume 6, Issue 3, November 1982, Pages 339-353. doi: 10.1016/0378-8741(82)90055-1

Camu camu (Myrciaria dubia) is a small bush that grows in the Amazon rainforest. The berries of the Camu camu bush contain a large amount of vitamin C, amino acids, trace minerals, vitamin B3, and potassium. Camu camu is consumed for its anti-oxidant and free radical scavenging properties. It has been considered in the treatment of diabetes. It may help improve muscle function and may fight inflammation. It may also support emotional stability and enhance mood. Langley, P., et al. (2015). Antioxidant and Associated Capacities of Camu Camu (Myrciaria dubia): A Systematic Review. J. Altern. Complement. Med. January 1; 21(1): 8-14. doi: 10.1089/acm.2014.0130

Astragalus is an herb that appears to protect the body against various stresses, including physical, mental, or emotional stress. It has been studied for potential blood sugar and blood pressure lowering effects. Astragalus may help prevent colds and seasonal allergy symptoms by supporting mucous membranes and epithelial cells in the respiratory tract. Astragalus as Astragalus propinquus or Astragalus membranaceus has been researched for its cardio protective, anti-inflammatory, immune response effects, and longevity effects. Tan, B. K. H., et al. (2004). Immunomodulatory and Antimicrobial Effects of Some Traditional Chinese Medicinal Herbs: A Review. Current Medicinal Chemistry, Volume 11, Number 11, June 2004, pp. 1423-1430(8). doi: 10.2174/0929867043365161

The mangosteen (Garcinia mangostana) is a tropical evergreen tree. It's edible fruit is an potent source of vitamin C it also contains minerals such as copper, manganese, magnesium, and potassium and B-complex vitamins such as thiamin, niacin, and folates. The fruit also contains xanthones, which have antioxidant free radical scavenging properties. Mangosteens and extracts from mangosteens may fight cancer, pain, allergies, infections, skin disorders, fatigue, and support intestinal health. Mangosteen may also be a natural and effective antihistamine. Gutierrez-Orozco, F., et al. (2013). Biological Activities and Bioavailability of Mangosteen Xanthones: A Critical Review of the Current Evidence. Nutrients, 2013, 5(8), 3163-3183. doi: 10.3390/nu5083163

Grape (Vitis vinifera) seed extract displays anti-oxidant properties. It may slow the aging process by offering free radical scavenging effects that maintain skin elasticity and collagen production. It may benefit conditions such as high cholesterol, atherosclerosis, high blood pressure, poor circulation and nerve damage. It also exhibits anti-inflammatory, anti-allergic, anti-cancerous and anti-microbial activity. Terra, X., et al. (2007). Grape-Seed Procyanidins Act as Antiinflammatory Agents in Endotoxin-Stimulated RAW 264.7 Macrophages by Inhibiting NFkB Signaling Pathway. Journal of Agricultural and Food Chemistry, 2007 55 (11), 4357-4365. doi: 10.1021/00633185

Turmeric (Curcuma longa) is a perennial plant has been used as a natural dye and food coloring and for its medicinal qualities. It has been used in both traditional Chinese medicine and Indian medicine. Turmeric contains curcuminoids, including curcumin, which are bioactive substance that exhibit anti-oxidant and anti-inflammatory effects. Turmeric may also enhance bodily enzymatic detoxification systems. Basnet, P., et al. (2011). Curcumin: An Anti-Inflammatory Molecule from a Curry Spice on the Path to Cancer Treatment. Molecules, 2011, 16(6), 4567-4598. doi:10.3390/molecules16064567.

Flax (Linum usitatissimum) seeds may help improve digestion, clear up the skin, lower cholesterol, reduce sugar cravings, balance hormones and promote weight loss. Flax Seeds contain both soluble and insoluble fibers and omega-3 essential fatty acids that have been shown to have effects that may benefit the heart. Flax seeds are high in magnesium and vitamin B1. Alpha-linolenic acid (ALA), a substance that is found in flaxseed and flaxseed oil, may benefit people with heart disease. The ALA in flax can help protect the lining of the digestive tract and maintain gastro-intestinal health. In the 8th century, King Charlemagne passed laws requiring people to consume flax seeds for their health benefits. Adolphe, J. L., et al. (2010). Health effects with consumption of the flax lignan secoisolariciresinol diglucoside. British Journal of Nutrition, 103(7), pp. 929-938. doi: 10.1017/S0007114509992753.

The unique quantities of the nutrient supplements combined in the present invention have been selected to provide a convenient nutrient supplement formulation that provides a broad scope of human and animal nutrition benefits.

The present invention is a nutritional supplement formulation comprising a combination of the nutrients biotin, calcium, a salt or bioavailable form of calcium, magnesium, a salt or bioavailable form of magnesium, selenium, a salt or bioavailable form of selenium, spirulina powder, chlorella powder, l-glutamine free acid or a salt of l-glutamine, acai berry powder, dandelion root extract powder, ginseng root extract powder, camu camu berry powder, astragalus root extract, mangosteen fruit extract powder, grape seed extract powder, turmeric root extract powder, ground flaxseed hulls, and further wherein the formulation has a total nutrient formulation weight consisting of the weight of all nutrients in the formulation.

The nutrients used in the present invention may alternatively be extracts of water, alcohol, or a mixture of alcohol and water that have been extracted in the same ratios as the water based extracts herein described and which have been further dried and standardized to the same amounts of flavonoids, mangostins, xanthones, polyphenols, curcuminoids, lignans, or other standardized active ingredient respectively for the herbal substance as herein described.

In one embodiment, the contemplated formulations of the present invention, are oral dose forms and the nutrient supplement composition of the invention may be formulated into any suitable oral dose form. Such suitable dose forms include but are not limited to capsules, gelatin capsules, hydroxypropyl methylcellulose capsules, soft-gel capsules, tablets, solutions, or the nutrients in combination with an edible mixture of an edible vehicle, food composition, or formed into, for example, a nutrient bar.

In one embodiment, the formulation of the present invention may further comprise the nutrients wherein, the salt of calcium is calcium carbonate, the salt of magnesium is magnesium citrate, the bioavailable form of selenium is l-selenomethionine, the spirulina powder is dried Arthrospira platensis powder, the chlorella powder is dried Chlorella vulgaris powder, the l-glutamine free acid or a salt of l-glutamine is l-glutamine free acid, the acai berry powder is dried Euterpe oleracea powder, the dandelion root extract powder is a dried 4:1 water, alcohol, or water and alcohol mixture extract of Taraxacum officinale, the ginseng root extract powder is a dried 5:1 water, alcohol, or water and alcohol mixture extract of Eleutherococcus senticosus, the Camu camu berry powder is dried and ground fruit of Myrciaria dubia, the astragalus root extract is a dried 4:1 water, alcohol, or water and alcohol mixture extract of Astragalus propinquus or Astragalus membranaceus, the mangosteen fruit extract powder is a dried 15:1 water, alcohol, or water and alcohol mixture extract of Garcinia mangostana fruit standardized to 95% flavonoids and 40% mangostins as xanthones, the grape seed extract powder is a dried water, alcohol, or water and alcohol mixture extract of Vitis vinifera standardized to 95% polyphenols, the turmeric root extract powder is a dried water, alcohol, or water and alcohol mixture extract of Curcuma longa standardized to 95% curcuminoids, and the flaxseed hulls are ground hulls of Linum usitatissimum standardized to 3.5% lignans.

The nutrient ingredients comprising the present invention include extracts of herbs and fruits, dried and ground natural substances, and mineral forms that may have various sizes of crystalline structures. There may be large variations in loft or volume of the nutrient ingredients, especially the herbal substances. In addition, the herbal substances may have varying quantities of the standardized or reference compounds that naturally occur in the nutrient ingredients. The varying quantities or concentrations of the standardized compounds, for example, polyphenols, in dried extracts may depend on the cultivation and growing practices used to cultivate the herbs. Therefore, there may be variations in the amount of bulk material for any nutrient ingredient used in the present invention. The relative amount of a nutrient ingredient may also depend on the extent of drying of the nutrient ingredient or extract of the nutrient ingredient. Therefore, in order to ensure that the formulation of ingredients may fit within a single dosage form capsule, the relative quantities for any single nutrient ingredient to be combined, in an embodiment of the formulation of the present invention, may vary from one-half to two times of an average weight for the single nutrient ingredient, with respect to an average of the total nutrient formulation weight.

Correspondingly, in one embodiment, the formulation of the present invention may further comprise the nutrients wherein the nutrients are in the following weight percentage ranges: the biotin is from 0.0361 to 0.144 percent of the total nutrient formulation weight, the calcium carbonate is from 0.724 to 2.90 percent of the total nutrient formulation weight, the magnesium citrate is from 0.724 to 2.90 percent of the total nutrient formulation weight, the l-selenomethionine is from 0.000361 to 0.00144 percent of the total nutrient formulation weight, the Arthrospira platensis powder is from 9.65 to 28.6 percent of the total nutrient formulation weight, the dried Chlorella vulgaris powder is from 9.65 to 28.6 percent of the total nutrient formulation weight, the l-glutamine free acid is from 4.827 to 19.3 percent of the total nutrient formulation weight, the dried Euterpe oleracea powder is from 4.827 to 19.3 percent of the total nutrient formulation weight, the dried 4:1 water, alcohol, or water and alcohol mixture extract of Taraxacum officinale is from 4.827 to 19.3 percent of the total nutrient formulation weight, the dried 5:1 water, alcohol, water and alcohol mixture extract of Eleutherococcus senticosus is from 3.61 to 14.4 percent of the total nutrient formulation weight, the dried fruit of Myrciaria dubia is from 3.61 to 14.4 percent of the total nutrient formulation weight, the dried 4:1 water, alcohol, or water and alcohol mixture extract of Astragalus propinquus or Astragalus membranaceus is from 2.41 to 9.66 percent of the total nutrient formulation weight, the dried 15:1 water, alcohol, or alcohol and water mixture extract of Garcinia mangostana fruit standardized to 95% flavonoids and 40% mangostins as xanthones is from 1.93 to 7.72 percent of the total nutrient formulation weight, the dried water, alcohol, or water and alcohol mixture extract of Vitis vinifera standardized to 95% polyphenols is from 1.086 to 4.34 percent of the total nutrient formulation weight, the dried water, alcohol, or water and alcohol mixture extract of Curcuma longa standardized to 95% curcuminoids is from 1.086 to 4.34 percent of the total nutrient formulation weight, and the dried and ground flaxseed hulls of Linum usitatissimum standardized to 3.5% lignans are from 0.965 to 3.86 percent of the total nutrient formulation weight.

In one embodiment, the formulation of the present invention may further comprise the nutrients wherein the nutrients are in the following percentage weight of the total nutrient weight formulation: the biotin is 0.0724 percent of the total nutrient formulation weight, the calcium carbonate is 1.448 percent of the total nutrient formulation weight, the magnesium citrate is 1.448 percent of the total nutrient formulation weight, the l-selenomethionine is 0.000724 percent of the total nutrient formulation weight, the Arthrospira platensis powder is 19.309 percent of the total nutrient formulation weight, the dried Chlorella vulgaris powder is 19.309 percent of the total nutrient formulation weight, the l-glutamine free acid is 9.655 percent of the total nutrient formulation weight, the dried Euterpe oleracea powder is 9.655 percent of the total nutrient formulation weight, the dried 4:1 water, alcohol, or water and alcohol mixture extract of Taraxacum officinale is 9.655 percent of the total nutrient formulation weight, the dried 5:1 water, alcohol, water and alcohol mixture extract of Eleutherococcus senticosus is 7.241 percent of the total nutrient formulation weight, the dried fruit of Myrciaria dubia is 7.241 percent of the total nutrient formulation weight, the dried 4:1 water, alcohol, or water and alcohol dried mixture of Astragalus propinquus or Astragalus membranaceus is 4.827 percent of the total nutrient formulation weight, the dried 15:1 water, alcohol, or alcohol and water mixture extract of Garcinia mangostana fruit standardized to 95% flavonoids and 40% mangostins as xanthones is 3.862 percent of the total nutrient formulation weight, the dried water, alcohol, or water and alcohol mixture extract of Vitis vinifera standardized to 95% polyphenols is 2.172 percent of the total nutrient formulation weight, the dried water, alcohol, or water and alcohol mixture extract of Curcuma longa standardized to 95% curcuminoids is 2.172 percent of the total nutrient formulation weight, and the ground flaxseed hulls of Linum usitatissimum standardized to 3.5% lignans are 1.931 percent of the total nutrient formulation weight.

In one embodiment, the formulation of the present invention may further comprise the nutrients wherein the weight of each nutrient is as follows: the biotin is 0.375 mg per the calcium carbonate is 7.5 mg per oral dose form, the magnesium citrate is 7.5 mg per oral dose form, the l-selenomethionine is 0.15 mg per oral dose form, the Arthrospira platensis powder is 400 mg per oral dose form, the dried Chlorella vulgaris powder is 400 mg per oral dose form, the l-glutamine free acid is 200 mg per oral dose form, the dried Euterpe oleracea powder is 200 mg per oral dose form, the dried 4:1 water, alcohol, or water and alcohol mixture extract of Taraxacum officinale is 200 mg oral dose form, the dried 5:1 water, alcohol, water and alcohol mixture extract of Eleutherococcus senticosus is 150 mg per oral dose form, the dried fruit of Myrciaria dubia is 150 mg per oral dose form, the dried 4:1 water, alcohol, or water and alcohol mixture extract of Astragalus propinquus or Astragalus membranaceus is of Astragalus propinquus and the dried extract of Astragalus propinquus is 100 mg per oral dose form, the dried 15:1 water, alcohol, or alcohol and water mixture extract of Garcinia mangostana fruit standardized to 95% flavonoids and 40% mangostins as xanthones is 80 mg per oral dose form, the dried water, alcohol, or water and alcohol mixture extract of Vitis vinifera standardized to 95% polyphenols is 45 mg per oral dose form, the dried water, alcohol, or water and alcohol mixture extract of Curcuma longa standardized to 95% curcuminoids is 45 mg per oral dose form, and the ground flaxseed hulls of Linum usitatissimum standardized to 3.5% lignans are 40 mg per capsule, oral dose form.

In one embodiment, the formulation of present invention may further comprise wherein the dried 4:1 water, alcohol, or water and alcohol mixture extract of Taraxacum officinale is a water extract, the dried 5:1 water, alcohol, water and alcohol mixture extract of Eleutherococcus senticosus is a water extract, the dried 4:1 water, alcohol, or water and alcohol mixture extract of Astragalus propinquus or Astragalus membranaceus is a water extract of Astragalus propinquus, the dried 15:1 water, alcohol, or alcohol and water mixture extract of Garcinia mangostana fruit standardized to 95% flavonoids and 40% mangostins as xanthones is a water extract, the dried water, alcohol, or water and alcohol mixture extract of Vitis vinifera standardized to 95% polyphenols is a water extract, and the dried water, alcohol, or water and alcohol mixture extract of Curcuma longa standardized to 95% curcuminoids is a water extract.

In one embodiment, the formulation of the present invention may further comprise hydroxypropyl methylcellulose, dicalcium phosphate, and l-leucine.

The variations in the elements of the invention will be readily understood by one of skill in the art to be interchangeable with the elements described herein. These descriptions of variations of elements of the present invention are intended to be exemplary and are not intended to be limiting in any way.

### Example 1

A representative formulation, which is suitable for an oral dose formulation of the nutritional supplement formulation of the present invention, was made as follows. A batch of 258,000 capsules of the present invention was made by first weighing out the nutrient supplement ingredients of the formulation in to a 55 cubic foot ribbon blender as follows, 5.491 kilograms of calcium carbonate, pharmaceutical grade, 13.545 kilograms of magnesium citrate, pharmaceutical grade, 25.800 kilograms of dried spirulina (Arthrospira platensis) powder, 25.800 kilograms of dried Chlorella (Chlorella vulgaris) powder, 13.545 kilograms of l-glutamine free acid, pharmaceutical grade, 12.900 kilograms of acai (Euterpe oleracea berry) powder, 12.900 kilograms of dried dandelion (Taraxacum officinale) root powder 4:1 water extract, 9.675 kilograms of dried Siberian ginseng (Eleutherococcus senticosus root) 5:1 water extract, 9.675 kilograms of dried Camu camu (Myrciaria dubia) berry powder, 6.450 kilograms of dried Astragalus (Astragalus propinquus) root 4:1 water extract, 5.160 kilograms of dried mangosteen (Garcinia mangostana) fruit 15:1 water extract, 3.048 kilograms dried water extract of grape (Vitis vinifera) seed standardized to 95% polyphenols by high performance liquid chromatography assay (HPLC), 2.903 kilograms of dried turmeric root (Curcuma longa root) water extract standardized to 95% curcuminoids by HPLC, ground dried flaxseed hulls (Linum usitatissimum) standardized to 3.5% lignans by HPLC, 12.900 kilograms of dicalcium phosphate, pharmaceutical grade, and 12.629 kilograms of l-leucine, pharmaceutical grade.

The weighed nutrient ingredients of the formulation were then mixed for 15 minutes in the 55 cubic foot ribbon blender, Charles Ross & Son Co., 710 Old Willets Path, Hauppauge, N.Y. 11788.

Then 0.106 kilograms of biotin and 0.203 kilograms of l-selenium-methionine, pharmaceutical grade, 2.345 kilograms of dicalcium phosphate and 2.345 kilograms of l-leucine were added to the existing mixture of nutrient supplement ingredients in the 55 cubic foot ribbon blender and the entire mixture was mixed for five minutes.

Then 10.555 additional kilograms of dicalcium phosphate and 10.284 additional kilograms of l-leucine were added to the mixture in the 55 cubic foot ribbon blender and mixed for ten additional minutes.

The mixture was then poured and loaded into a fully automated capsule filler machine, AF-90T ACG, ACG North America LLC, 229 Durham Ave., South Plainfield, N.J. 07080, or 127, ACG Worldwide, Kandivli Industrial Estate, Kandivli (West), Mumbai 400 067, India, which had been previously loaded with hydroxypropyl methylcellulose (vegetarian capsules) size #00. The capsules were loaded and filled capsule weight calculated and averaged.

The variance in weight of the filled capsule formulation is shown in Table 1, Master Manufacturing and Batch Record. The yield tolerance per capsule is +/−10%.

I claim:

1. A nutritional supplement formulation comprising the nutrients: biotin, calcium, a salt or bioavailable form of calcium, magnesium, a salt or bioavailable form of magnesium, selenium, a salt or bioavailable form of selenium, spirulina powder, chlorella powder, l-glutamine free acid or a salt of l-glutamine, acai berry powder, dandelion root extract powder, ginseng root extract powder, camu camu berry powder, astragalus root extract, mangosteen fruit extract powder, grape seed extract powder, turmeric root extract powder, ground flaxseed hulls, and further wherein the formulation has a total nutrient formulation weight consisting of the weight of all nutrients in the formulation.

2. The formulation of claim 1 wherein the formulation is an oral dose form comprising a capsule, gelatin capsule, hydroxypropyl methylcellulose capsule, soft-gel capsule, tablet, pellet, sachet, solution, or the nutrients in combination with an edible mixture of a vehicle or food composition.

3. The nutritional supplement formulation of claim 2 wherein,
the salt of calcium is calcium carbonate,
the salt of magnesium is magnesium citrate,
the bioavailable form of selenium is l-selenomethionine,
the spirulina powder is dried Arthrospira platensis powder,
the chlorella powder is dried Chlorella vulgaris powder,
the l-glutamine free acid or a salt of l-glutamine is l-glutamine free acid,
the acai berry powder is dried Euterpe oleracea powder,
the dandelion root extract powder is a dried 4:1 water, alcohol, or water and alcohol mixture extract of Taraxacum officinale,
the ginseng root extract powder is a dried 5:1 water, alcohol, or water and alcohol mixture of Eleutherococcus senticosus,
the camu camu berry powder is dried fruit of Myrciaria dubia,
the astragalus root extract is a dried 4:1 water, alcohol, or water and alcohol mixture extract of Astragalus propinquus or Astragalus membranaceus,
the mangosteen fruit extract powder is a dried 15:1 water, alcohol, or water and alcohol mixture extract of Garcinia mangostana fruit standardized to 95% flavonoids and 40% mangostins as xanthones,
the grape seed extract powder is a dried water, alcohol, or water and alcohol mixture extract of Vitis vinifera standardized to 95% polyphenols,
the turmeric root extract powder is a dried water, alcohol, or water and alcohol mixture extract of Curcuma longa standardized to 95% curcuminoids, and
the ground flaxseed hulls are of Linum usitatissimum standardized to 3.5% lignans.

4. The nutritional supplement formulation of claim 3 wherein the nutrients are in the following weight percentage ranges:
the biotin is from 0.0361 to 0.144 percent of the total nutrient formulation weight,
the calcium carbonate is from 0.724 to 2.90 percent of the total nutrient formulation weight,
the magnesium citrate is from 0.724 to 2.90 percent of the total nutrient formulation weight,
the l-selenomethionine is from 0.000361 to 0.00144 percent of the total nutrient formulation weight,
the Arthrospira platensis powder is from 9.65 to 28.6 percent of the total nutrient formulation weight,
the dried Chlorella vulgaris powder is from 9.65 to 28.6 percent of the total nutrient formulation weight,
the l-glutamine free acid is from 4.827 to 19.3 percent of the total nutrient formulation weight,
the dried Euterpe oleracea powder is from 4.827 to 19.3 percent of the total nutrient formulation weight,
the dried 4:1 water, alcohol, or water and alcohol mixture extract of Taraxacum officinale is from 4.827 to 19.3 percent of the total nutrient formulation weight,
the dried 5:1 water, alcohol, water and alcohol mixture extract of Eleutherococcus senticosus is from 3.61 to 14.4 percent of the total nutrient formulation weight,
the dried fruit of Myrciaria dubia is from 3.61 to 14.4 percent of the total nutrient formulation weight,
the dried 4:1 water, alcohol, or water and alcohol mixture extract of Astragalus propinquus or Astragalus membranaceus is from 2.41 to 9.66 percent of the total nutrient formulation weight,
the dried 15:1 water, alcohol, or alcohol and water mixture extract of Garcinia mangostana fruit standardized to 95% flavonoids and 40% mangostins as xanthones is from 1.93 to 7.72 percent of the total nutrient formulation weight,
the dried water, alcohol, or water and alcohol mixture extract of Vitis vinifera standardized to 95% polyphenols is from 1.086 to 4.34 percent of the total nutrient formulation weight,
the dried water, alcohol, or water and alcohol mixture extract of Curcuma longa standardized to 95% curcuminoids is from 1.086 to 4.34 percent of the total nutrient formulation weight, and
the ground flaxseed hulls of Linum usitatissimum standardized to 3.5% lignans are from 0.965 to 3.86 percent of the total nutrient formulation weight.

5. The nutritional supplement formulation of claim 4 wherein the nutrients are in the following percentage weight of the total nutrient weight formulation:
the biotin is 0.0724 percent of the total nutrient formulation weight,
the calcium carbonate is 1.448 percent of the total nutrient formulation weight,
the magnesium citrate is 1.448 percent of the total nutrient formulation weight,
the l-selenomethionine is 0.000724 percent of the total nutrient formulation weight,
the Arthrospira platensis powder is 19.309 percent of the total nutrient formulation weight,
the dried Chlorella vulgaris powder is 19.309 percent of the total nutrient formulation weight,
the l-glutamine free acid is 9.655 percent of the total nutrient formulation weight,
the dried Euterpe oleracea powder is 9.655 percent of the total nutrient formulation weight,
the dried 4:1 water, alcohol, or water and alcohol mixture extract of Taraxacum officinale is 9.655 percent of the total nutrient formulation weight,
the dried 5:1 water, alcohol, water and alcohol mixture extract of Eleutherococcus senticosus is 7.241 percent of the total nutrient formulation weight,
the dried fruit of Myrciaria dubia is 7.241 percent of the total nutrient formulation weight,
the dried 4:1 water, alcohol, or water and alcohol mixture extract of Astragalus propinquus or Astragalus membranaceus is 4.827 percent of the total nutrient formulation weight,
the dried 15:1 water, alcohol, or alcohol and water mixture extract of Garcinia mangostana fruit standardized to 95% flavonoids and 40% mangostins as xanthones is 3.862 percent of the total nutrient formulation weight,
the dried water, alcohol, or water and alcohol mixture extract of Vitis vinifera standardized to 95% polyphenols is 2.172 percent of the total nutrient formulation weight,
the dried water, alcohol, or water and alcohol mixture extract of Curcuma longa standardized to 95% curcuminoids is 2.172 percent of the total nutrient formulation weight, and
the ground flaxseed hulls of Linum usitatissimum standardized to 3.5% lignans are 1.931 percent of the total nutrient formulation weight.

6. The formulation of claim 5 further comprising wherein the
the biotin is 0.375 mg per oral dose form,
the calcium carbonate is 7.5 mg per oral dose form,
the magnesium citrate is 7.5 mg per oral dose form,
the l-selenomethionine is 0.15 mg per oral dose form,
the Arthrospira platensis powder is 400 mg per oral dose form,
the dried Chlorella vulgaris powder is 400 mg per oral dose form,
the l-glutamine free acid is 200 mg per oral dose form,
the dried Euterpe oleracea powder is 200 mg per oral dose form,
the dried 4:1 water, alcohol, or water and alcohol mixture extract of Taraxacum officinale is 200 mg per oral dose form,
the dried 5:1 water, alcohol, water and alcohol mixture extract of Eleutherococcus senticosus is 150 mg per oral dose form,
the dried fruit of Myrciaria dubia is 150 mg per oral dose form,
the dried 4:1 water, alcohol, or water and alcohol mixture extract of Astragalus propinquus or Astragalus membranaceus is Astragalus propinquus and the Astragalus propinquus is 100 mg per oral dose form,
the dried 15:1 water, alcohol, or alcohol and water mixture extract of Garcinia mangostana fruit standardized to 95% flavonoids and 40% mangostins as xanthones is 80 mg per oral dose form,
the dried water, alcohol, or water and alcohol mixture extract of Vitis vinifera standardized to 95% polyphenols is 45 mg per oral dose form,
the dried water, alcohol, or water and alcohol mixture extract of Curcuma longa standardized to 95% curcuminoids is 45 mg per oral dose form, and
the ground flaxseed hulls of Linum usitatissimum standardized to 3.5% lignans are 40 mg per oral dose form.

7. The formulation of claim 6 further comprising hydroxypropyl methylcellulose, dicalcium phosphate, and l-leucine.

8. The formulation of claim 6 further wherein the dried 4:1 water, alcohol, or water and alcohol mixture extract of Taraxacum officinale is a water extract,
the dried 5:1 water, alcohol, water and alcohol mixture extract of Eleutherococcus senticosus is a water extract,
the dried 4:1 water, alcohol, or water and alcohol mixture extract of Astragalus propinquus or Astragalus membranaceus is a water extract of Astragalus propinquus,
the dried 15:1 water, alcohol, or alcohol and water mixture extract of Garcinia mangostana fruit standardized to 95% flavonoids and 40% mangostins as xanthones is a water extract,
the dried water, alcohol, or water and alcohol mixture extract of Vitis vinifera standardized to 95% polyphenols is a water extract, and
the dried water, alcohol, or water and alcohol mixture extract of Curcuma longa standardized to 95% curcuminoids is a water extract.
